(19) United States
(12) Patent Application Publication
(10) Pub. No.: US 2014/0276559 A1
(43) Pub. Date: Sep. 18, 2014

(54) DEVICES, SYSTEMS AND METHODS FOR PLACEMENT OF INSTRUMENTS FOR MEDICAL PROCEDURES

(71) Applicant: Christopher Page, Rye Brook, NY (US)

(72) Inventor: Christopher Page, Rye Brook, NY (US)

(21) Appl. No.: 13/795,345

(22) Filed: Mar. 12, 2013

(51) Int. Cl. A61B 17/34

(52) U.S. Cl. CPC A61B 17/3403
USPC 604/506; 604/272

(57) ABSTRACT

Medical guide devices, systems and methods are provided, comprising a base member and a rotatable medical guide component seated on the base member. The guide component defines a path extending through the guide component. The guide component includes a first radio-opaque marker located at a first end of the guide component corresponding with an entry point of the path and a second radio-opaque marker located at a second end of the guide component corresponding with an exit point of the path, and the second end is opposite the first end.

### FIELD

The present disclosure relates to devices, systems and methods for accurate placement of medical instruments during medical procedures, including fluoroscopy needle guide devices, systems and methods.

### BACKGROUND

Medical procedures often require real time X-ray guidance, known as fluoroscopy, to position needles or other devices. In such procedures, it is very important that the positioning of these devices be accurate. Accuracy of positioning is typically determined by aligning the needle path parallel to the incident angle of the X-ray beam. To do this, a medical practitioner has to take multiple fluoroscopic images while advancing the needle. After each image is taken, the needle is adjusted in an attempt to obtain a “gun-barrel” view of the needle.

This view is consistent with the needle following a path exactly parallel to the X-ray beam and appears as a single dot on the X-ray image. Following this path, the needle will, at a given depth from the surface, reach a target that has previously been aligned with the X-ray emitter and detector. This technique typically requires manual adjustment of the needle with only the previous X-ray image for guidance, and existing devices for facilitating needle guidance have several disadvantages.

The subsequent re-orientation of the X-ray beam, to appreciate the needle in different trajectories, deprives the operator of the ability to perceive if the needle has continued to advance accurately in the original path. Also, multiple X-rays are required to confirm that the needle remains in this path throughout advancement, exposing the medical practitioner to additional radiation. Moreover, once most existing needle guide devices are placed on a patient, adjustment to align the guide markers with target is limited by the fixed guide holes of the devices. Finally, for most existing devices, once the needle is inserted into the guide, the guide cannot be moved or removed unless the needle is withdrawn.

In addition, other medical devices used to facilitate the accurate placement of medical instruments and devices during medical procedures suffer from similar drawbacks. Such instruments might include surgical hardware such as surgical screws and pins, radiofrequency and cryoablative probes, drains, catheters, ventriculostomies and chest tubes.

Accordingly, there is a need for a medical instrument positioning system that facilitates accurate placement of those instruments. There is a need for a needle guide device, system and method that facilitates fine adjustment prior to insertion of the needle. There is also a need for a needle guide device, system and method that does not require the operator to reposition the device on the patient and use additional electromagnetic radiation. Finally, there is a need for needle guide devices, systems and methods that allow for freedom of movement and provide better accuracy while also allowing for the device to be removed while leaving the needle in position if necessary.

### SUMMARY

The present disclosure, in its many embodiments, alleviates to a great extent the disadvantages of known devices, systems and methods for placement of medical instruments during medical procedures, particularly needle guide devices, systems and methods, by providing a substantially spherical needle guide device seated in a base member wherein the needle guide has two radio-opaque markers at opposite ends around an opening that allows passage of a needle through the needle guide and the markers. The disclosed devices, systems and methods advantageously facilitate accurate and fine adjustment prior to insertion of the needle while reducing radiation exposure and obviate the need to reposition the device on the patient.

Exemplary embodiments include a medical guide device comprising a base member and a rotatable guide component seated on the base member. The guide component defines a path extending therethrough. The guide component includes a first radio-opaque marker located at a first end of the guide component corresponding with an entry point of the path and a second radio-opaque marker located at a second end of the guide component corresponding with an exit point of the path. A needle may be provided to be inserted through the path. The second end of the guide component is opposite the first end. The guide component may be substantially spherical.

In exemplary embodiments, the first and second radio-opaque markers appear in parallel alignment relative to each other when the path is aligned with a surgical target and an X-ray beam. One or both of the guide component and the base member may be composed of a radio-lucent material. In exemplary embodiments, a side surface of the guide component defines a channel therein. The medical guide device may further comprise an attachment mechanism to releasably secure the guide component to the base member.

Exemplary embodiments include methods of using a guide component comprising providing a base member, providing a rotatable guide component, seating the guide component partially within the base member, and rotating the guide component within the base member. The guide component defines a path extending therethrough and includes a first radio-opaque marker located at a first end of the guide component corresponding with an entry point of the path and a second radio-opaque marker located at a second end of the guide component corresponding with an exit point of the path. The second end of the guide component is opposite the first end. The guide component is rotated within the base member such that the first and second radio-opaque markers appear in parallel alignment relative to each other. The guide component may be substantially spherical and may be made of a radio-lucent material.

In exemplary methods, the rotating step comprises aligning the path with a surgical target and an X-ray beam. The aligning step may comprise positioning the path parallel to an incident angle of the X-ray beam. Exemplary methods may further comprise inserting a needle through the path. In exemplary methods, the base member is made of a radio-lucent material.

In exemplary embodiments, a rotatable guide component comprises a substantially spherical component defining a path extending therethrough. The guide component includes a first radio-opaque marker located at a first end of the substantially spherical component corresponding with an entry point of the path and a second radio-opaque marker located at a second end of the substantially spherical component corresponding with an exit point of the path. The second end of the rotatable guide component is opposite the first end. The substantially spherical component is freely positionable at multiple angles, and the first and second radio-opaque markers appear in parallel alignment relative to each other when the path is aligned with a surgical target and an X-ray beam.

In exemplary embodiments, the rotatable guide component further comprises a base member, and the substantially spherical component is seated on the base member. The first and second radio-opaque markers may appear in parallel alignment relative to each other when the path is positioned parallel to an incident angle of the X-ray beam. The rotatable guide component may further comprise a needle inserted through the path, and the needle may remain in a consistent trajectory. In exemplary embodiments, one or both of the substantially spherical component and the base member is composed of a radio-lucent material.

Accordingly, it is seen that medical guide devices, systems and methods are provided which allow accurate and fine adjustment prior to insertion of the medical instrument and obviate the need to reposition the device on the patient. These and other features of the present disclosure will be appreciated from review of the following detailed description of exemplary embodiments, along with the accompanying figures in which like reference numbers refer to like parts throughout.

### BRIEF DESCRIPTION OF THE DRAWINGS

The foregoing and other objects of the disclosure will be apparent upon consideration of the following detailed description, taken in conjunction with the accompanying drawings, in which:

FIG. 1 is a schematic of an existing fluoroscopy system;

FIG. 2A is a schematic of an existing fluoroscopic needle insertion method;

FIG. 2B is a top view of an existing fluoroscopic needle insertion method;

FIG. 2C is a top view of an existing fluoroscopic needle insertion method;

FIG. 3A is a side cutaway view of an embodiment of a medical guide device in accordance with the present disclosure;

FIG. 3B is a side cutaway view of an embodiment of a medical guide device in accordance with the present disclosure;

FIG. 4A is a side cutaway view of an embodiment of a medical guide device in accordance with the present disclosure;

FIG. 4B is a side cutaway view of an embodiment of a medical guide device in accordance with the present disclosure;

FIG. 5A is a top view of the medical guide device of FIG. 4A;

FIG. 5B is a top view of the medical guide device of FIG. 4B;

FIG. 6 is a top view of an embodiment of a needle guide device in accordance with the present disclosure; and

FIG. 7 is a perspective view of an embodiment of a medical guide device in accordance with the present disclosure.

### DETAILED DESCRIPTION

In the following paragraphs, embodiments will be described in detail by way of example with reference to the accompanying drawings, which are not drawn to scale, and the illustrated components are not necessarily drawn proportionately to one another. Throughout this description, the embodiments and examples shown should be considered as exemplars, rather than as limitations of the present disclosure. As used herein, the “present disclosure” refers to any one of the embodiments described herein, and any equivalents. Furthermore, reference to various aspects of the disclosure throughout this document does not mean that all claimed embodiments or methods must include the referenced aspects. Reference to temperature, pressure, density and other parameters should be considered as representative and illustrative of the capabilities of exemplary embodiments, and embodiments can operate with a wide variety of such parameters. It should be noted that the figures do not show every piece of equipment, nor the pressures, temperatures and flow rates of the various streams.

As shown in FIG. 1, fluoroscopy generally involves positioning needles 10a, 10b or other devices for medical procedures to reach a target site 4 in a patient 6. A fluoroscope 18 includes an X-ray detector 8 and an X-ray emitter 16 that emits X-ray beams 12. The X-ray beams 12 travel through a patient 6 and is detected by X-ray detector 8. To position the needles 10a, 10b accurately, the needle path is typically aligned parallel to the incident angle 14 of the X-ray beam 12. The medical practitioner must take multiple fluoroscopic images while advancing the needle and adjust the needle manually after each image to maintain the needle in parallel alignment with the X-ray beam. FIG. 2A shows an example in which needle 10a is in parallel alignment with the X-ray beam 12, but needle 10b is in non-parallel alignment. FIG. 2B shows a “gun-barrel” view in which the needle 10a is in proper parallel alignment. FIG. 2C shows needle 10b in non-parallel alignment. As discussed above, these current methods of fluoroscopy have significant disadvantages including the need for manual adjustment of the needle, multiple re-orientations of the needle, multiple X-rays, and inaccuracy in needle placement.

Turning to FIGS. 3A and 3B, embodiments of the present disclosure will be described which alleviate these problems with existing fluoroscopy methods. A medical guide device 20 includes a rotatable guide component 22, which may be any shape that allows it to freely rotate. In exemplary embodiments, the medical guide device 20 is a needle guide device, and the guide component 22 is a substantially spherical, or ball-shaped, needle guide. The needle guide 22 defines a path 24 that runs internally through the needle guide 22 from a first end 26 to a second end 28 opposite the first end. The path 24 defines an entry point 30 at the first end 26 of the needle guide 22 and an opposite exit point 32 at the second end 28 of the needle guide 22. As discussed in more detail herein, a needle 10 is also provided for insertion through the needle path 24.

Exemplary embodiments of a needle guide device 20 utilize a system of radio-opaque and radio-lucent materials to facilitate the positioning of radio-opaque objects during fluoroscopic procedures. As discussed in detail herein, these markers and materials advantageously provide more accurate placement of needles with less radiation exposure. Moreover, they maintain the proper needle trajectory even when the imaging angle of the fluoroscope is changed during a procedure. As seen in FIGS. 3A and 3B, the needle guide 22 includes radio-opaque markers 34, 36. A first radio-opaque marker 34 is located at the first end 26 of the needle guide 22 and is positioned at the entry point 30 of the needle path 24. Opposite the first marker 34 is a second radio-opaque marker 36 at the second end 28 of the needle guide, positioned at the exit point 32 of the needle path 24.

The markers 34, 36 could be made of any radio-opaque materials, including, but not limited to, metals such as aluminum, stainless steel, or titanium, as well as any other material or combination of materials capable of obstructing X-rays. As discussed in more detail herein, the radio-opaque markers 34, 36 are designed and positioned so they assume a specific orientation relative to one another when the needle path 24 is aligned with the medical practitioner's surgical target and the X-ray beam.

The needle guide device 20 also includes a base member 38 having a size and shape that corresponds with the needle guide 22 so the needle guide 22 can be seated at least partially within the base member 38. In exemplary embodiments in which the needle guide 22 is substantially spherical, the base member 38 is substantially concave to accommodate a bottom portion of the needle guide 22 in a close fitting and stable seating arrangement. With the needle guide 22 seated in the base member 38, the needle guide device 20 can be placed on the operating surface of a patient 6. The bottom surface 44 of the base member 38 could be coated with an adhesive material 46 so the needle guide device 20 could be positioned on non-flat surfaces of the patient and still be operational. A needle access opening 45 is defined in the bottom surface 44 of the base member 38 to allow the needle 10 to exit the needle guide device 20 and enter the patient 6. The needle access opening 45 should be large enough to allow the needle guide 22 sufficient room to rotate and still permit the needle 10 to extend out of the exit point 32 of the needle path 24 and into the patient 6 but should not be larger than the diameter of the needle guide 22.

An attachment mechanism 42 may also be provided to secure the needle guide 22 to the base member 38. In particular, the attachment mechanism 42 could be one or more clips 42a, 42b, which can releasably secure the needle guide 22 to the base member 38 in a way that provides ease of attachment, rotation of the needle guide 22 when seated, and release and removal of the needle guide 22. As best seen in FIG. 7, two flexible clips 42a, 42b are attached to the base member 38 to hold the needle guide 22 in place while allowing it to freely rotate. Clips 42 are malleable enough to allow removal of the needle guide 22 if it became necessary to remove the device without removing the needle. Advantageously, this could be accomplished without the need for a needle channel in the base member 38 itself as it would have a large enough aperture to be removed alone without the guide in place. As discussed in more detail herein, in exemplary embodiments the attachment mechanism 42 allows the needle guide 22 to be freely moved or rotated while seated in the base member 38 and secured when the desired guide position is obtained.

The needle guide 22, the base member 38, or both components could be made of a radio-lucent material. Any material or combination of materials that are transparent or transradiant to electromagnetic radiation, i.e., permit the passage of X-rays, can be used, including, but not limited to, polymers such as plastics and thermoplastic resins, or carbon and carbon-fiber composites. More particularly, in exemplary embodiments the only radio-opaque portions of the needle guide device 20 are the markers 34, 36 while the remainder of the needle guide 22 and base member 38 are made of completely radio-lucent materials, or materials that are radio-lucent relative to the markers 34, 36 so as not to obscure detection of a needle or underlying structures.

Referring to FIG. 7, an exemplary embodiment of a rotatable guide component 22 defines a channel 23 cut into its side so the medical guide device 20 can be removed without withdrawing a needle from the patient. More particularly, a channel 23 may be cut in parallel to the entry point 30 of the path 24. Optionally, a base channel 25 could be cut into the base member 38 to further ease removal of the rotatable guide component 22 from the base member 38. When the base channel 25 is aligned with the channel 23 in the rotatable guide component 22, the base channel 25 would provide an opening that would allow removal of the guide device 20 without altering the position of the needle or other instrument being positioned. In exemplary embodiments, a removable or detachable component along the needle channel could be provided to act as a channel guard to prevent the movement of the guide device inadvertently before the needle has been completely positioned.

It should be noted that embodiments of the device could be used to facilitate the accurate placement of other medical instruments and devices. Any device that requires the use of fluoroscopic guidance could be improved by the use of this method both in terms of accuracy and minimizing radiation exposure to the patient and operator. This could include but is not limited to surgical hardware such as surgical screws and pins, radiofrequency and cryoablative probes, drains, catheters, ventriculostomies and chest tubes.

In operation, a medical practitioner can use exemplary embodiments in any application where accurate fluoroscopic guidance is required, particularly, where radio-opaque objects need to be accurately position relative to deep structures. Exemplary embodiments are useful in a number of medical settings, including, but not limited to, needle placement for tissue biopsy, needle placement of medication injection, needle placement for ablative therapy, percutaneous device implantation, and orthopedic hardware insertion. First, the operator places the base member 38 on the surface of a patient 6. Then, the operator seats the needle guide 22 in the base member 38. Alternatively, the operator may seat the needle guide 22 in the base member 38 first and then place the complete needle guide device 20 on the surface of the patient 6. The needle guide 22 may be releasably secured to the base member 38 using attachment mechanism 42.

As shown in FIGS. 4A and 4B, once the needle guide device 20 is properly positioned on the patient and the X-ray beam is projecting on the device, the operator rotates the needle guide 22 in the base member 38 until the needle guide 22 is positioned properly. More particularly, the operator rotates the needle guide 22 until that the exit point 32 of the needle path 24 is aligned with the target site 4 of the patient and X-ray beam 12. A particular advantage of disclosed embodiments is that the first and second radio-opaque markers 34, 36 assume a specific orientation to one another when this alignment is achieved.

As best seen in FIGS. 5A and 5B, the proper orientation of the first and second radio-opaque markers 34, 36, indicating the correct alignment, is readily apparent to the operator. When the needle path 24 of the needle guide 22 is aligned parallel to the incident angle 14 of the X-ray beam 12 and the target site 4 of the patient, as shown in FIG. 4A, the first and second radio-opaque markers 34, 36 are in an eclipse-type orientation such that they overlay each other, which can be best seen in FIG. 5A. This depiction shown in FIG. 5A is the view seen by the operator. By contrast, when the needle path 24 is in non-parallel orientation and out of alignment with the X-ray beam 12 and the target site of the patient, as shown in FIG. 4B, the first and second radio-opaque markers 34, 36 are seen by the operator as being in two different locations, as best seen in FIG. 5B. Thus, it is readily apparent to the operator when the needle guide device 20 is aligned properly and when it is not.

When the operator sees that the first and second radio-opaque markers 34, 36 are in an eclipse-type or overlay orientation, he or she knows that the needle path 24 is properly positioned exactly parallel to the incident angle 14 of the X-ray beam 12 and properly aligned with the surgical target of the patient. Then the operator secures the needle guide 22 within the base member 38 using the attachment mechanism 42 so the needle guide 22 is locked in the aligned position. With reference to FIG. 6, the operator then inserts the needle 10 through the needle path 24, through the needle access opening 45, and into the patient 6 to reach the target site.

Advantageously, the needle guide device 20 facilitates advancement of the needle 10 in a consistent trajectory to the target site of the patient. Moreover, no additional X-ray images from the aligned position are required so the operator can reposition the X-ray beam 12 to observe the needle 10 from other angles without compromising the original needle trajectory. This advantageously allows the operator to determine the proper depth of needle placement by imaging from a second, non-parallel angle. If desired, the operator can then remove both the needle guide 22 and the base member 38 of the needle guide device 20 from the patient 6 while leaving the needle 10 in the patient 6 at the target site. If necessary, the operator can repeat the procedure to insert additional needles into the patient.

Thus, it is seen that fluoroscopic needle guide devices and methods are provided. It should be understood that any of the foregoing configurations and specialized components or chemical compounds may be interchangeably used with any of the systems of the preceding embodiments. Although illustrative embodiments are described hereinabove, it will be evident to one skilled in the art that various changes and modifications may be made therein without departing from the disclosure. It is intended in the appended claims to cover all such changes and modifications that fall within the true spirit and scope of the disclosure.

What is claimed is:

1. A medical guide device comprising:
a base member; and
a rotatable guide component seated on the base member, the guide component defining a path extending therethrough;
the guide component including a first radio-opaque marker located at a first end of the guide component corresponding with an entry point of the path and a second radio-opaque marker located at a second end of the guide component corresponding with an exit point of the path, the second end being opposite the first end.

2. The device of claim 1 wherein the first and second radio-opaque markers appear in parallel alignment relative to each other when the path is aligned with a surgical target and an X-ray beam.

3. The device of claim 1 wherein the guide component is substantially spherical.

4. The device of claim 1 wherein one or both of the guide component and the base member is composed of a radio-lucent material.

5. The device of claim 1 wherein a side surface of the guide component defines a channel therein.

6. The device of claim 1 further comprising an attachment mechanism to releasably secure the guide component to the base member.

7. The device of claim 1 further comprising a needle inserted through the path.

8. A method of using a guide component, comprising:
providing a base member;
providing a rotatable guide component, the guide component defining a path extending therethrough and including a first radio-opaque marker located at a first end of the guide component corresponding with an entry point of the path and a second radio-opaque marker located at a second end of the guide component corresponding with an exit point of the path, the second end being opposite the first end
seating the guide component partially within the base member; and
rotating the guide component within the base member such that the first and second radio-opaque markers appear in parallel alignment relative to each other.

9. The method of claim 8 wherein the rotating step comprises aligning the path with a surgical target and an X-ray beam.

10. The method of claim 9 wherein the aligning step comprises positioning the path parallel to an incident angle of the X-ray beam.

11. The method of claim 10 further comprising inserting a needle through the path.

12. The method of claim 8 wherein the guide component provided is substantially spherical.

13. The method of claim 8 further comprising making the guide component of a radio-lucent material.

14. The method of claim 8 further comprising making the base member of a radio-lucent material.

15. A rotatable medical guide comprising:
a substantially spherical component defining a path extending therethrough and including a first radio-opaque marker located at a first end of the substantially spherical component corresponding with an entry point of the path and a second radio-opaque marker located at a second end of the substantially spherical component corresponding with an exit point of the path, the second end being opposite the first end;
wherein the substantially spherical component is freely positionable at multiple angles and the first and second radio-opaque markers appear in parallel alignment relative to each other when the path is aligned with a surgical target and an X-ray beam.

16. The rotatable medical guide of claim 15 further comprising a base member;
wherein the substantially spherical component is seated on the base member.

17. The rotatable medical guide of claim 16 wherein the first and second radio-opaque markers appear in parallel alignment relative to each other when the path is positioned parallel to an incident angle of the X-ray beam.

18. The rotatable medical guide of claim 17 further comprising a needle inserted through the path.

19. The rotatable medical guide of claim 18 wherein the needle remains in a consistent trajectory.

20. The rotatable medical guide of claim 16 wherein one or both of the substantially spherical component and the base member is composed of a radio-lucent material.
